(19) United States
(12) Patent Application Publication
(10) Pub. No.: US 2026/0267050 A1
(43) Pub. Date: Sep. 10, 2026

(54) LIGHT DIFFUSION DEVICE

(71) Applicant: FURUKAWA ELECTRIC CO., LTD., Tokyo (JP)

(72) Inventors: Keisuke TAKE, Tokyo (JP); Hideaki HASEGAWA, Tokyo (JP); Kyosuke YAMAUCHI, Tokyo (JP); Kumi KAWASHIMA, Tokyo (JP); Junichi HASEGAWA, Tokyo (JP)

(73) Assignee: FURUKAWA ELECTRIC CO., LTD., Tokyo (JP)

(21) Appl. No.: 19/167,494

(22) PCT Filed: Mar. 22, 2024
(86) PCT No.: PCT/JP2024/011288

§ 371 Date: Sep. 22, 2025

(30) Foreign Application Priority Data
Mar. 31, 2023 (JP) 2023-059367

(51) Int. Cl. F21V 8/00

(52) U.S. Cl. CPC G02B 6/001

(57) ABSTRACT

A light diffusion device causing light that has entered from a proximal-end part of the optical fiber to be output from a distal-end side of the optical fiber. The optical fiber includes an optical transmission part and a light output part for allowing the light transmitted through the light transmission part to be output from an outer peripheral surface on the distal-end side. The light output part is treated to suppress heat generation, and includes a reflection member having a light reflectance of 80% or more in an area other than an area constituting a light output opening on the outer peripheral surface of the light output part, and in a distal-end surface. The reflection member includes particles having a light reflecting property, the particles including barium sulfate.

### TECHNICAL FIELD

The present invention relates to a light diffusion device for use in medical equipment.

### BACKGROUND ART

There is known a conventional light diffusion device (diffuser) that is provided with an optical fiber including: a core located on a center side in a radial direction; and a cladding located on an outer peripheral side of the core, in which the laser light introduced from a proximal end portion BE of the optical fiber is emitted from a distal end portion T and an outer peripheral surface on a distal end side T of the optical fiber (see, for example, Patent Document 1). The optical fiber of the conventional light diffusion device includes: a light transmission section configured to transmit the laser light introduced from the proximal end portion BE; and a light emission section configured to emit the laser light transmitted through the light transmission section from the distal side T.

The light diffusion device is used in photodynamic therapy (PDT) and photoimmunotherapy (PIT), which are cancer treatment methods using light. In such therapies, the distal side T of the optical fiber is inserted into the human body, and laser light is used to irradiate a drug that has been administered and has reached cancer cells. The diffuser emits light laterally from the distal end portion T of the optical fiber. Normally, light is emitted in the circumferential direction, which raises concerns that the light may reach not only cancer cells but also healthy cells.

### CITATION LIST

### Patent Document

Patent Document 1: Japanese Unexamined Patent Application (Translation of PCT Application), Publication No. 2012-506569

### DISCLOSURE OF THE INVENTION

### Problems to be Solved by the Invention

For example, in treating metastatic liver cancer, especially in the case of a small tumor, there is a demand for providing the distal end of the optical fiber with a cylindrical diffuser for insertion into the affected area, and limiting the irradiation angle and area to irradiate only the affected area as much as possible. Accordingly, there is a technique of using a reflective member (shielding element) that surrounds the outer peripheral surface of the light emission section, leaving a region serving as a light emission port. Of the light emitted radially outward from the outer peripheral surface of the light emission section, the portion directed to a region other than the light emission port is reflected by the reflective member and redirected toward the light emission port. As a result, the light can be limited to a predetermined region in the circumferential direction, allowing strong light to irradiate only the affected area.

However, the reflective member may cause a problem of generating heat at and near the light emission section. When the heat generated at the light emission section of the light diffusion device becomes significant, there is a concern that the patient as the subject of treatment may perceive heat, or the heat may affect healthy cells. Therefore, from the perspectives of improving efficiency, ease, and precision of procedure, a light diffusion device capable of suppressing heat generation at and near the light emission section is desired.

It is one of the objects of the present invention to provide a light diffusion device capable of suppressing heat generation at and near the light emission section while limiting the irradiation region.

### Means for Solving the Problems

A light diffusion device according to one aspect of the present invention includes: an optical fiber including a core located on a center side in a radial direction, and a cladding located on an outer peripheral side of the core, in which the light diffusion device is configured to emit, from a distal side of the optical fiber, light introduced from a proximal end portion of the optical fiber. The optical fiber is made of plastic and includes: a light transmission section configured to transmit the light from the proximal end portion to a distal end portion; and a light emission section configured to emit the light transmitted through the light transmission section from an outer peripheral surface on a distal side, in which the light emission section is subjected to a treatment for suppressing heat generation. A region other than a region serving as a light emission port on the outer peripheral surface of the light emission section, and a distal end surface, are provided with a reflective member having a light reflectance of 80% or more. The reflective member includes light-reflective particles. The particles include barium sulfate.

One aspect of the present invention may further provide a protective member configured to cover and seal the reflective member.

According to one aspect of the present invention, at least a part of the cladding in the light emission section may be removed. In this case, a rough surface may be formed on an outer peripheral surface, from which the cladding in the light emission section is removed, and at least one selected from a group consisting of Rp, Rz, and Ra, which are indicators of surface roughness of the outer peripheral surface, may be less than or equal to a wavelength of light emitted from the light emission section.

According to one aspect of the present invention, the reflective member extending in an axial direction may be provided in an arc-shaped region having a predetermined central angle θ on an outer peripheral surface of the optical fiber, and a remaining arc-shaped region not provided with the reflective member may serve as the light emission port. In this case, the central angle θ may be 180° or more.

One aspect of the present invention may further provide a light-non-transmissive first sheath configured to surround at least the light emission section of the optical fiber,
an opening may be provided in a region of the first sheath, such that the opening can be positioned to face the light emission port, and
the optical fiber may be rotatable relative to the first sheath in a circumferential direction.

One aspect of the present invention may further provide a light-non-transmissive first sheath configured to surround at least the light emission section of the optical fiber,
an opening may be provided in a region of the first sheath, such that the opening can be positioned to face the light emission port, and
the optical fiber may be movable relative to the first sheath in an axial direction. This case may further provide a light-transmissive balloon-shaped second sheath, outside the first sheath.

### Effects of the Invention

According to one embodiment of the present invention, it is possible to provide a light diffusion device capable of suppressing heat generation at and near the light emission section while limiting the irradiation region.

### BRIEF DESCRIPTION OF THE DRAWINGS

FIG. 1 is a schematic diagram of a light diffusion device according to a first embodiment as an exemplary aspect of the present invention;

FIG. 2 is a cross-sectional view of a plane including an axis x of a light emission section and its vicinity of an optical fiber in the light diffusion device according to the first embodiment, and corresponds to the cross-sectional view taken along the line B-B in FIG. 3;

FIG. 3 is a cross-sectional view of a plane perpendicular to the axis x at a portion of the light emission section of the optical fiber in the light diffusion device according to the first embodiment, and corresponds to the cross-sectional view taken along the line A-A in FIG. 2;

FIG. 4 is an enlarged cross-sectional view illustrating a main portion of the optical fiber in the light diffusion device according to the first embodiment;

FIG. 5 is a cross-sectional view of a plane including an axis x of a light emission section and its vicinity of an optical fiber in a light diffusion device according to a second embodiment as another exemplary aspect of the present invention, and corresponds to the cross-sectional view taken along the line D-D in FIG. 6;

FIG. 6 is a cross-sectional view of a plane perpendicular to the axis x at a portion of the light emission section of the optical fiber in the light diffusion device according to the second embodiment, and corresponds to the cross-sectional view taken along the line C-C in FIG. 5;

FIG. 7 is a perspective view of the light emission section and its vicinity of the optical fiber in the light diffusion device according to the second embodiment;

FIG. 8 is a cross-sectional view of the same section as FIG. 6, illustrating an example in which the light emission (irradiation) region in the circumferential direction is narrowed in the light diffusion device according to the second embodiment;

FIG. 9 is a cross-sectional view of the same section as FIG. 5, illustrating an example in which the light emission (irradiation) region in the axial direction is changed in the light diffusion device according to the second embodiment;

FIG. 10 is another cross-sectional view of the same section as FIG. 5, illustrating another example in which the light emission (irradiation) region in the axial direction is changed in the light diffusion device according to the second embodiment;

FIG. 11 is a cross-sectional view of a plane including an axis x of a light emission section and its vicinity of an optical fiber in a light diffusion device according to a third embodiment as another exemplary aspect of the present invention; and

FIG. 12 is a perspective view of the light emission section and its vicinity of the optical fiber in the light diffusion device according to the third embodiment.

### PREFERRED MODE FOR CARRYING OUT THE INVENTION

Hereinafter, three embodiments of the light diffusion device according to exemplary aspects of the present invention will be described in detail with reference to the drawings.

### First Embodiment

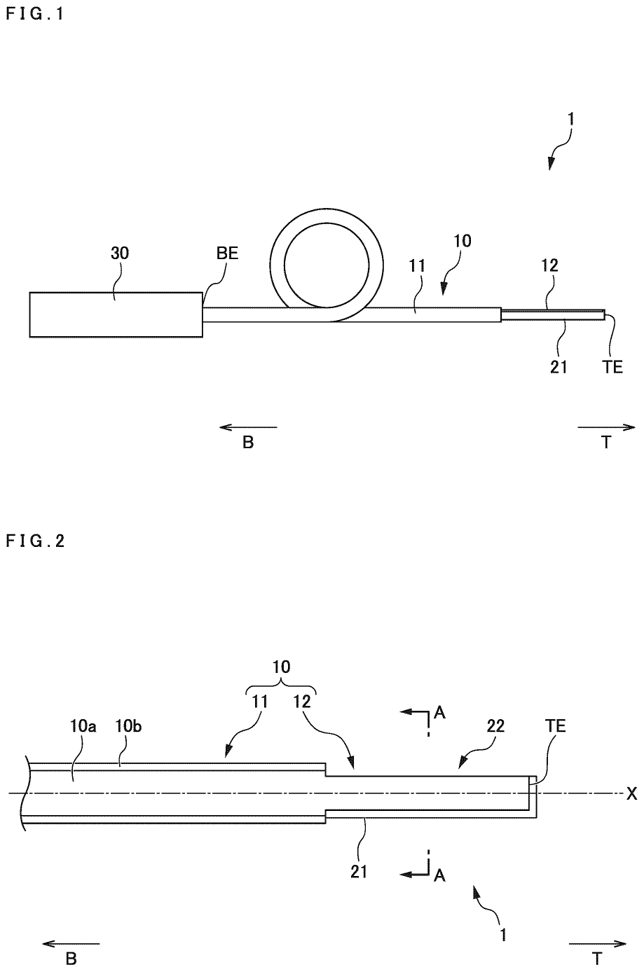

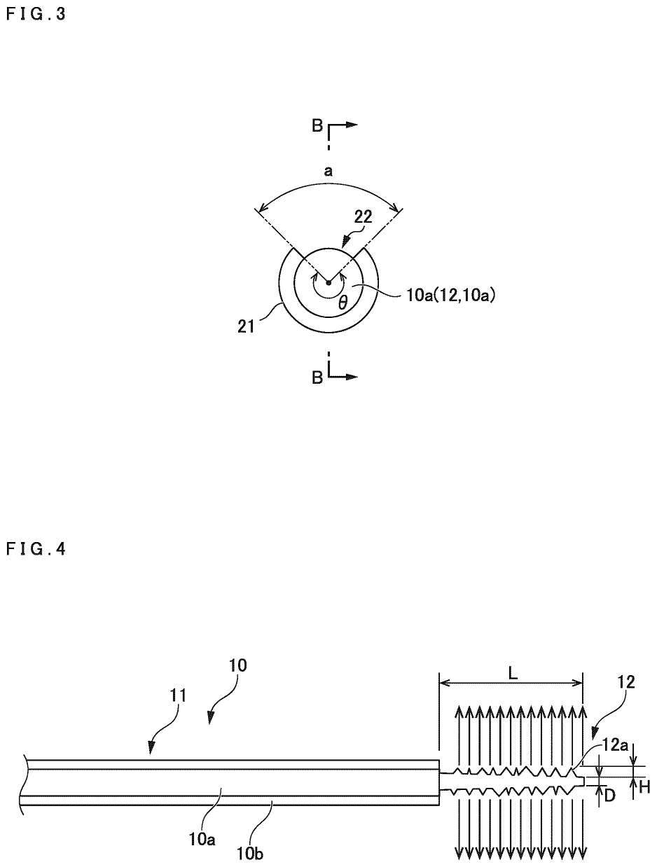

FIG. 1 is a schematic diagram of a light diffusion device 1 according to the first embodiment; FIG. 2 is a cross-sectional view of a plane including an axis x of a light emission section 12 and its vicinity of an optical fiber in the light diffusion device 1; and FIG. 3 is a cross-sectional view of a plane perpendicular to the axis x at a portion of the light emission section 12. Specifically, FIG. 2 corresponds to the cross-sectional view taken along the line B-B in FIG. 3; and FIG. 3 corresponds to the cross-sectional view taken along the line A-A in FIG. 2. In the drawings, an arrow B indicates a proximal side B of the optical fiber, and an arrow T indicates a distal side T thereof.

The light diffusion device 1 of the present embodiment is used in photodynamic therapy (PDT) and photoimmunotherapy (PIT), which are cancer treatment methods. In particular, photoimmunotherapy is a procedure in which a drug composed of an antibody capable of binding to cancer cells and a substance capable of reacting to light is administered to the human body, and laser light is used to irradiate the drug bound to the cancer cells, thereby destroying the cancer cells.

As illustrated in FIG. 1, the light diffusion device 1 is a device configured to emit, from a distal side T, light introduced from a proximal end portion BE of an optical fiber 10, in which a laser oscillator 30 serving as a light source for generating laser light is connected to the proximal end portion BE on the proximal side B of the optical fiber 10.

The laser oscillator 30 includes a semiconductor laser and generates laser oscillation by supplying electricity to the semiconductor laser, thereby generating laser light. The laser oscillator 30 generates laser light having a wavelength between 500 nm and 900 nm inclusive.

The optical fiber 10 is made of a plastic (resin) member. As illustrated in FIG. 3, the optical fiber 10 is a single-core optical fiber that includes: a core 10a located on a center side in a radial direction; and a cladding 10b located on an outer peripheral side of the core 10a.

The relative refractive index difference between the core 10a and the cladding 10b in the optical fiber 10 is between 2% and 11% inclusive. For example, the outer diameter of the optical fiber 10 is 500 μm, the outer diameter of the core 10a is 480 μm, and the thickness of the cladding 10b is 10 μm. Here, it is preferable that the outer diameter of the cladding 10b of the optical fiber 10 be between 102 μm and 1100 μm inclusive. In the optical fiber 10, it is preferable that the outer diameter of the core 10a be between 100 μm and 1000 μm inclusive. It is preferable that the cladding 10b have a thickness between 1 μm and 50 μm inclusive.

As illustrated in FIGS. 1 and 2, the optical fiber 10 includes: a light transmission section 11 configured to transmit laser, toward the distal side T, light introduced from the proximal end portion BE; and a light emission section 12 configured to emit the laser, from an outer peripheral surface, light that is transmitted through the light transmission section 11, the light emission section 12 being formed by removing the cladding 10b within a predetermined range to the distal side T in an extending direction.

As illustrated in FIGS. 2 and 3, a reflective member 21 is provided on a portion of the outer peripheral surface of the light emission section 12 and on a distal end surface. Specifically, as illustrated in FIG. 3, the reflective member 21 extends in an axial direction x in an arc-shaped region having a predetermined central angle θ on the outer peripheral surface of the optical fiber 10. A remaining arc-shaped region a, in which the reflective member 21 is not provided, serves as a light emission port 22. The reflective member 21 blocks and further reflects light that would be emitted from the outer peripheral surface of the light emission section 12 other than from the light emission port 22, so that the light is emitted from the light emission port 22.

In the present embodiment, the central angle θ defining the region in which the reflective member 21 is provided, is set to 120°, for example. In light of the objective of limiting the irradiation (emission) range, the central angle θ is preferably 180° or more (i.e., the region a serving as the irradiation range is 180° or less), and more preferably 210° or more (i.e., the region a serving as the irradiation range is 150° or less).

The light reflectance of the reflective member 21 is preferably 80% or more, more preferably 90% or more, and still more preferably 95% or more. By using the reflective member 21 having high light reflectance, it is possible to limit the irradiation region and emit light with high efficiency, while suppressing heat generation at and near the light emission section 12. It is noted that the term “light reflectance” herein includes both specular reflection and diffuse reflection.

The reflective member 21 is preferably formed as thin as possible. By thinning the reflective member 21, the diameter at the light emission section 12 of the light diffusion device 1 (i.e., the diameter constituted by the optical fiber 10 and the reflective member 21 at the light emission section 12) can be reduced. As a result, it becomes possible to insert the device into a thin needle catheter, pass it through narrow blood vessels in the body, or access organs through such blood vessels, thereby improving procedural flexibility.

It is preferable that the diameter at the light emission section 12 of the light diffusion device 1 be 1 mm or less. By setting the diameter at the light emission section 12 of the light diffusion device 1 to 1 mm or less, the device can be inserted into a general needle catheter. In the second embodiment described later, a general needle catheter may be modified and used as a first sheath.

Specifically, the thickness of the reflective member 21 is preferably 500 nm or less, and more preferably 300 nm or less. On the other hand, although thinner is generally better for the lower limit of the thickness of the reflective member 21, excessively thin thickness may not retain the desired light reflectance. Accordingly, it is preferable to make the reflective member as thin as possible while ensuring the desired light reflectance.

Even if the reflective member 21 has a thickness exceeding the preferred range described above, although the advantages of thinning the reflective member 21 may not be achieved, the effect of suppressing heat generation at and near the light emission section 12 while limiting the irradiation region may still be realized. In such a case, the reflective member 21 may be formed not in a layered structure but, for example, in a solid mass.

The material of the reflective member 21 is not particularly limited as long as the above conditions of light reflectance can be satisfied; however, the reflective member 21 preferably includes light-reflective particles, allowing for achieving high light reflectance even in a thin film. By forming the reflective member 21 using individual light-reflective particles, it is possible to achieve higher light reflectance with a very thin film.

Examples of the light-reflective particles preferably used as a material for the reflective member 21 include barium sulfate, titanium oxide, silica, alumina, calcium carbonate, and various porous materials (e.g., porous polytetrafluoroethylene (PTFE) ). Among these, barium sulfate, titanium oxide, silica, and alumina are preferred, and barium sulfate is particularly preferred due to an extremely high light reflectance (approximately 97%-98%).

The reflective member 21 may be formed by applying a coating liquid prepared by mixing the light-reflective particles with an adhesive resin such as polyvinyl alcohol, epoxy resin, or silicone resin, and a solvent such as water or alcohol, to a region to be coated, and then drying and curing the applied film. In this case, in the circumferential direction, for example, a seal may be applied in advance to the region a where the reflective member 21 is not to be provided, the above coating liquid may be applied thereto, and after drying and curing, the seal may be removed to define the light emission port 22.

In the case where the reflective member 21 is formed using light-reflective particles, a protective member (not illustrated) may be provided to cover and seal the reflective member 21 so that the formed layer is not detached. By sealing the reflective member 21 with the protective member, detachment of the reflective member 21 can be prevented. Furthermore, in cases where the reflective member 21 does not have biocompatibility, the reflective member 21 can be isolated using a protective member having biocompatibility.

The protective member may seal and cover the entire reflective member 21 or may be a tube that seals the entire circumference of the optical fiber 10 including the light emission port 22. In the case of using a tubular protective member, it is preferable to seal an end portion on the distal side T and to fill the inside with air to maintain an airtight state. The material of the protective member is not particularly limited as long as the material is a soft resin, and examples of the material include nylon, polyamide, PTFE, ethylene-tetrafluoroethylene copolymer (ETFE), polyvinyl chloride, polyvinylidene chloride, urethane resin, and silicone resin. Among these, nylon and silicone resin are particularly preferred due to the excellent biocompatibility.

As in the second or third embodiment described later, in a configuration in which a first sheath 23 (and further, a second sheath 26) is provided outside at least the light emission section 12 of the optical fiber 10, the protective member may be omitted.

Next, the optical fiber 10 will be described. FIG. 4 is an enlarged cross-sectional view illustrating a main portion of the optical fiber 10. The light emission section 12 is formed at a length L from the distal end T of the optical fiber 10, the length L being, for example, between 10 mm and 100 mm inclusive. The light emission section 12 is a portion where the cladding 10b has been removed to expose the core 10a, the portion extending over at least part of the distal side T of the optical fiber 10 in the circumferential direction and over a certain length in the axial direction of the distal side T of the optical fiber 10. In the present embodiment, the cladding 10b has been removed to expose the core 10a, in a portion of the distal side T of the optical fiber 10 in the circumferential direction.

A rough surface is formed as a treatment for light leakage on at least a part of the outer peripheral surface of the exposed core 10a. This rough surface serves as an uneven portion 12a having a height difference that is not greater than the wavelength of the laser light transmitted through the light transmission section 11. In the present embodiment, the uneven portion 12a is formed over the entire surface of the exposed core 10a.

The uneven portion 12a is formed on the outer peripheral surface of the core 10a by, for example, applying an etching process. As a treatment for suppressing heat generation, it is preferable that the height difference between peaks and valleys of the uneven portion 12 a be within a range of 0.1 times to 1 time the wavelength of the laser light transmitted through the light transmission section 11. The periodicity in uneven shape of peaks and valleys of the uneven portion 12a is equal to or smaller than the wavelength of the laser light transmitted through the light transmission section 11.

The uneven portion 12a is formed such that the height difference H is greater on the distal side T than on the proximal side B of the light emission section 12. Meanwhile, a radial dimension D of a portion of the core 10a in the light emission section 12, excluding the uneven portion 12a, is formed smaller on the distal side T than on the proximal side B of the light emission section 12. In the present embodiment, the height difference H in the uneven portion 12a gradually increases from the proximal end portion BE to the distal end portion T of the light emission section 12, and the radial dimension D of the portion of the core 10a excluding the uneven portion 12a gradually decreases from the proximal end portion BE to the distal end portion T of the light emission section 12. The radial dimension D of a portion of the core 10a in the light emission section 12, excluding the uneven portion 12a, is, for example, 75% at the distal end T relative to that at the proximal end B of the light emission section 12.

In the optical fiber 10, the light emission section 12 exposes the core 10a and includes a rough surface formed on the core, making it possible to emit laser light uniformly from the entire outer peripheral surface of the exposed core 10a. Since the light emission section 12 is formed by exposing the core 10a, it is possible to emit laser light with the required output, even when the height difference in the uneven portion 12a is small. Since the area of the interface between the outer peripheral surface of the core 10a in the light emission section 12 and air is reduced, thermal resistance at the interface can be decreased, and the amount of heat generation can be reduced.

Since the height difference of the uneven portion 12a in the light emission section 12 is greater on the distal side T than on the proximal side B of the light emission section 12, laser light can be more readily emitted from the distal side of the light emission section 12, making it possible to emit laser light more uniformly over the entire axial direction of the light emission section 12.

The radial dimension of the portion of the core 10a in the light emission section 12, excluding the uneven portion 12a, is smaller on the distal side T than on the proximal side B. In the present embodiment, in the light emission section 12, the distal end portion T is, for example, 75% of the size of the proximal end portion B. Therefore, the amount of laser light emitted from the distal end TE of the optical fiber 10 can be reduced.

Since the uneven portion 12a with a height difference equal to or less than the wavelength of the light transmitted through the light transmission section 11 is formed on at least part of the outer peripheral surface of the exposed core 10a, it is possible to emit laser light uniformly from the light emission section 12 of the optical fiber 10, and to reduce the amount of heat generated when the laser light is emitted. Thus, laser light can be emitted efficiently from the light emission section 12.

Since the periodicity in uneven shape of the uneven portion 12a is equal to or less than the wavelength of the laser light transmitted through the light transmission section 11, the emission amount in the light emission section 12 can be increased.

Regarding the surface roughness of the rough surface (uneven portion 12a) formed on at least part of the outer peripheral surface of the light emission section 12, it is preferable that the outer peripheral surface have at least one value selected from a group consisting of Rp (maximum peak height of a roughness profile), Rz (maximum height roughness), and Ra (arithmetical mean roughness), each of which is an indicator of surface roughness, and that the selected value be less than or equal to the wavelength of the light emitted from the light emission section 12. Note that Rp, Rz, and Ra are defined in Japanese Industrial Standard JIS B0601: 2013.

By forming the outer peripheral surface of the light emission section 12 into a rough surface (uneven portion 12a) having the appropriate surface conditions as described above, it is possible to suppress heat generation at and near the light emission section 12. With respect to the surface roughness indicators Rp, Rz, and Ra, it is sufficient if at least one of the surface roughness indicators is less than or equal to the wavelength of the light (emitted light) emitted from the light emission section 12. However, it is more preferable if two of the surface roughness indicators are less than or equal to the wavelength, and most preferable if all three of the surface roughness indicators are less than or equal to the wavelength.

In the light diffusion device 1 according to the present embodiment, when the laser oscillator 30 is activated and laser light is introduced from the proximal end portion BE of the optical fiber 10, the laser light is transmitted through the light transmission section 11 and emitted from the light emission section 12. The laser light emitted from the light emission section 12 of the optical fiber 10 toward the distal end TE and radially in all directions around the circumference is partially emitted directly from the light emission port 22, and the remainder is reflected by the reflective member 21 and then emitted from the light emission port 22. That is, the laser light transmitted through the light transmission section 11 is concentrated within a certain circumferential range in the light emission section 12 and emitted laterally from the optical fiber 10. Therefore, the transmitted laser light can efficiently irradiate an irradiation target such as cancer cells.

Generally, when laser light is emitted from the light emission port, heat tends to be generated near the light emission section of the optical fiber. Therefore, it is preferable for a practitioner to ensure that the temperature near the light emission section does not rise, for example, more than 5° C. above the body temperature of the subject. On the other hand, in order to achieve a high procedural effect and high time efficiency, a certain level of laser light intensity is desirable. Specifically, an output of approximately 400 mW/cm is preferable.

Continuous irradiation for a relatively long time with the above-described laser light intensity is desirable; however, heat generation at and near the light emission section 12 needs to be suppressed for that purpose. In the present embodiment, the light emission section 12 has been subjected to the above-described heat suppression treatment and includes the reflective member 21 having a very high light reflectance of 80% or more, thereby achieving suppression of heat generation at and near the light emission section 12.

In order to achieve a very high light reflectance of 80% or more, the particle diameter of the barium sulfate becomes an important factor. The average particle diameter of general commercial barium sulfate is approximately between 0.3 μm and 15 μm. As a layer for efficiently reflecting laser light, it is desirable that the barium sulfate particles be densely packed with small gaps between particles. The layer may be composed of barium sulfate having an average particle diameter within a relatively narrow range (for example, 0.3 μm to 0.5 μm), or within a wide range (for example, 0.3 μm to 15 μm) . Here, the term “average particle diameter” refers to the particle diameter (volume-based average particle diameter) at which the cumulative volume reaches 50% in a particle size distribution measured by the laser diffraction scattering method. The mixing ratio of barium sulfate having different average particle diameters, the dispersion state of barium sulfate having different average particle diameters, and the thickness of the barium sulfate layer should preferably be optimized as appropriate through experiments or the like to achieve the desired reflectance.

The reflective member 21 may be formed by applying a reflective coating (for example, a flexible film in which barium sulfate particles are dispersed in silicone resin), or by forming a reflective film (for example, a polyethylene terephthalate (PET) film coated with silicone resin containing barium sulfate), or by forming the reflective member 21 into a flexible or non-flexible plate shape or three-dimensional shape. By forming the reflective member in this manner, it becomes easier to dispose the reflective member 21 on the region other than the region serving as the light emission port 22 on the outer peripheral surface of the light emission section 12, and on the distal end surface.

The inventors fabricated a light diffusion device having the same configuration as the light diffusion device 1 of the present embodiment, using the reflective member 21 composed of barium sulfate particles with a light reflectance of 98%, and verified the heat generation characteristics. Specifically, when the output of the laser oscillator 30 was set to 400 mW/cm and irradiation was performed for 10 minutes, the temperature increased from an initial temperature of 24.2° C. to a maximum of 27.6° C., resulting in a temperature rise of only 2.4° C. In contrast, in a comparative example using a reflective film (formed by coating silicone resin containing titanium oxide on PET) as the reflective member, a temperature rise of 21.1° C. was observed under the same conditions, increasing from an initial temperature of 23.9° C. to a maximum of 55.0°C. Therefore, it was confirmed that the present embodiment significantly reduces heat generation.

### Second Embodiment

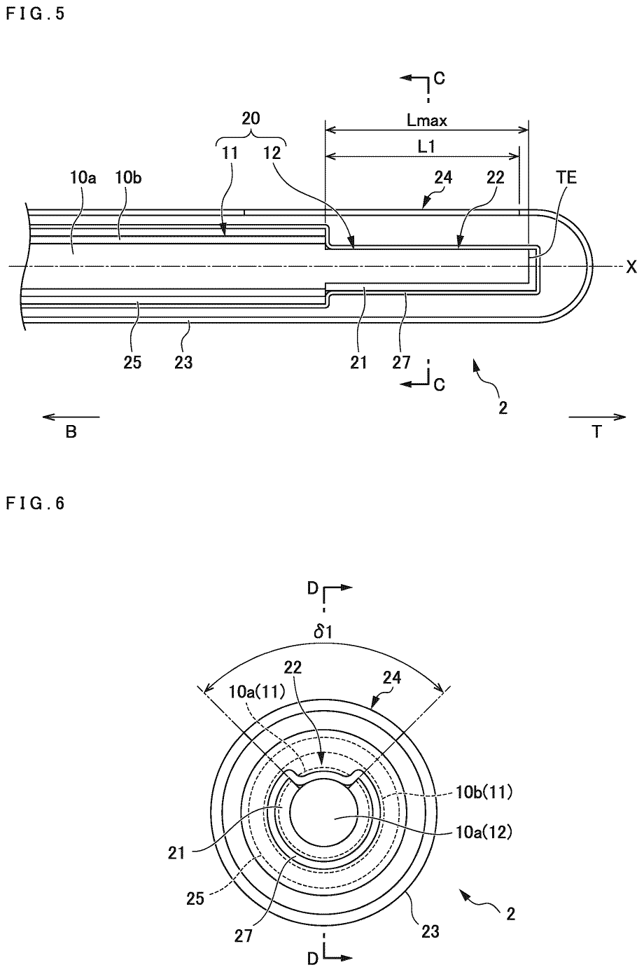

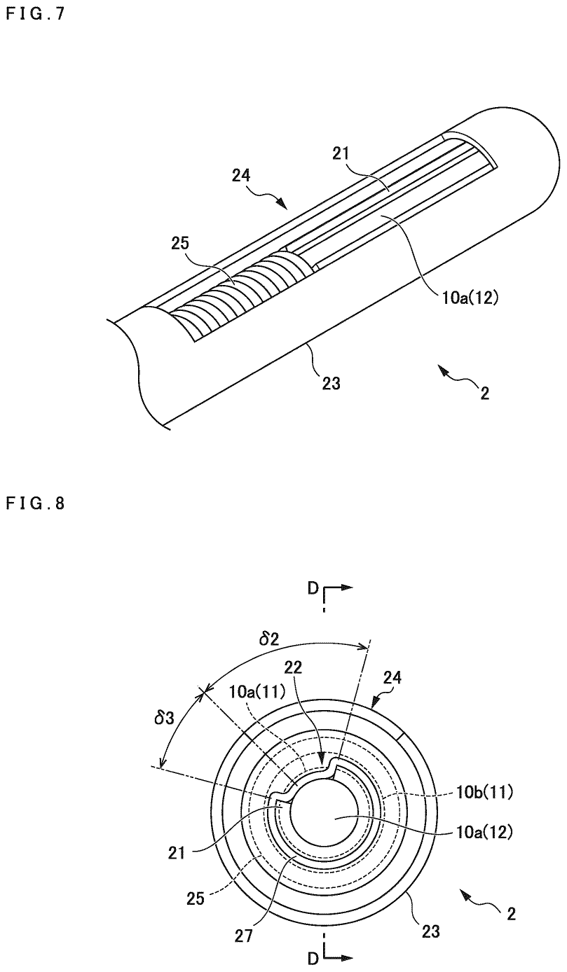

FIG. 5 is a cross-sectional view of a plane including an axis x of a light emission section 12 and its vicinity of the optical fiber 10 in a light diffusion device 2 according to the second embodiment; and FIG. 6 is a cross-sectional view of a plane perpendicular to the axis x at a portion of the light emission section 12. Specifically, FIG. 5 corresponds to a cross section taken along the line C-C in FIG. 6; and FIG. 6 corresponds to a cross section taken along the line D-D in FIG. 5. FIG. 7 is a perspective view of the light emission section 12 and its vicinity of the optical fiber 10 in the light diffusion device 2. Since the light diffusion device 2 of the present embodiment has substantially the same configuration as the light diffusion device 1 of the first embodiment, see FIG. 1 for an overview of the light diffusion device 2.

The light diffusion device 2 according to the second embodiment has the same configuration as the light diffusion device 1 of the first embodiment, except that a first sheath 23 is added. Therefore, the components having the same functions as those in the light diffusion device 1 of the first embodiment are designated with the same reference numerals as those in FIGS. 1 to 4, and detailed descriptions are omitted.

As illustrated in FIGS. 5 to 7, the light diffusion device 2 of the present embodiment includes a light-transmissive protective sheath 27 and a light-non-transmissive first sheath 23, both of which surround at least the light emission section 12 of the optical fiber 10. The protective sheath 27 corresponds to the member described as the “protective member” in the first embodiment, and uses, for example, a nylon tube.

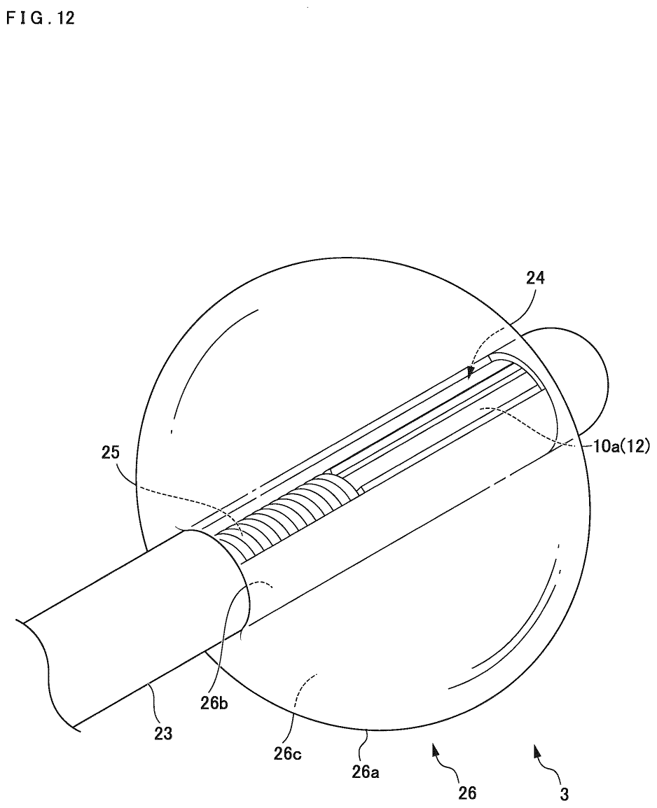

As illustrated in FIGS. 5 and 6, the protective sheath 27 follows the stepped structure of the layers while forming a gentle curved surface. However, the material for the protective sheath (protective member) may not necessarily have to be flexible to follow the stepped structure, and may have, for example, a flat shape that does not follow the stepped structure. As long as the member serves as the above-described protective member, the protective member only needs to have at least the function of covering and sealing the reflective member 21, regardless of whether the protective member follows the stepped structure. Note that in FIG. 7, the protective sheath 27 is omitted from illustration (the same applies to FIG. 12 of the third embodiment described later).

On the other hand, the first sheath 23 is generally referred to as a catheter, and is usually a cylindrical medical tube with a diameter of 20 mm or less or a rectangular medical tube with a side length of 20 mm or less. An opening 24 is provided in a region of the first sheath 23, such that the opening can be positioned to face the light emission port 22. The opening 24 is a rectangular opening corresponding to an arc-shaped region 81 having approximately the same central angle (i.e., 360°−θ) as the arc-shaped region a that serves as the light emission port 22. The central angle corresponding to the region where the light emission port 22 exists does not necessarily match the central angle corresponding to the region where the opening 24 exists. However, it is preferable that both of the central angles be the same, or that the central angle corresponding to the region δ1 where the opening 24 exists be equal to or greater than the central angle corresponding to the region where the light emission port 22 exists.

In order to make the first sheath 23 light-non-transmissive, the first sheath 23 itself may be made of a light-non-transmissive material, or a light-non-transmissive layer may be formed on the inner or outer peripheral surface of a sheath made of a light-transmissive material. In the case of forming the light-non-transmissive layer, it is particularly effective to form the layer with a metal such as stainless steel, which not only blocks the light but also actively reflects the light, ultimately allowing the light to be emitted from the opening 24 and thereby improving efficiency.

The opening 24 may be formed as a window-like region in which the first sheath 23 does not exist rectangularly, or may be formed by using a light-transmissive material only for that region. In a configuration in which a second sheath 26 is provided outside the first sheath 23, as in the third embodiment described later, the opening 24 may be a window-like region with no material present. However, in the case where the device of the present embodiment is used in a procedure, or in the case where the sealing performance of the second sheath 26 is insufficient, it is preferable to seal the interior and exterior of the first sheath 23 in order to protect the light emission port 22 of the optical fiber 10 and the reflective member 21.

In order to seal the interior and exterior of the first sheath 23, the window-like region of the opening 24 may be formed of a light-transmissive material, or a sealing tube may be fitted over the entire opening 24 and sealed, and the end on the distal side T of the tube may be closed. By sealing the interior and exterior of the first sheath 23, the interior of the first sheath 23 can be filled with air, and the light emission port 22 of the optical fiber 10 and the reflective member 21 can be protected.

In the light diffusion device 2 according to the present embodiment, the optical fiber 10 is rotatable relative to the first sheath 23 in the circumferential direction.

As illustrated in FIGS. 5 to 7, the light diffusion device 2 of the present embodiment includes a torque coil 25 wound around the light transmission section 11 of the optical fiber 10. The torque coil 25 is a metal coating (e.g., stainless steel) that imparts torque transmission capability to the optical fiber 10 and particularly restricts the rotational orientation (twist) of the optical fiber 10.

In the light diffusion device 2 according to the present embodiment, by providing the torque coil 25, a rotational operation of the optical fiber 10 relative to the first sheath 23 outside the body of the subject can be transmitted as the same rotational operation inside the body. The torque coil 25 may be omitted when a material having torque transmission capability is used for the optical fiber 10 itself.

In order to identify the relative position in the circumferential direction of the opening 24 of the first sheath 23 inside the body, it is preferable to place a mark in advance at a position in the circumferential direction of either the optical fiber 10 or the first sheath 23 outside the body.

As illustrated in FIG. 6, in the state where the position of the light emission port 22 of the optical fiber 10 and the position of the opening 24 of the first sheath 23 coincide in the circumferential direction, light is emitted from the entire region 81 in which the opening 24 exists. That is, in the present embodiment, light is emitted from the widest circumferential region 81 and irradiates the affected area.

From the state illustrated in FIG. 6, when a practitioner rotates the optical fiber 10 relative to the first sheath 23 outside the body of the subject, the light emission (irradiation) region in the circumferential direction can be narrowed. FIG. 8 is a cross-sectional view of the same section as FIG. 6 and illustrates an example in which the light emission (irradiation) region in the circumferential direction is narrowed, in the light diffusion device 2 according to the present embodiment.

FIG. 8 illustrates an example in which the optical fiber 10 is rotated counterclockwise, as viewed in FIG. 6, relative to the first sheath 23, from the state illustrated in FIG. 6. In the state illustrated in FIG. 8, in the circumferential direction, the light emitted from the light emission port 22 of the optical fiber 10 includes light corresponding to a certain region 53 on the left side, which is blocked by the first sheath 23, and only the light corresponding to the region 82 is emitted from the opening 24. Therefore, in the state illustrated in FIG. 8, the light emission (irradiation) region in the circumferential direction is narrowed.

In the light diffusion device 2 according to the present embodiment, the optical fiber 10 is configured to be movable relative to the first sheath 23 in the axial direction x, both toward the distal side T and toward the proximal side B. In order to identify the relative position of the opening 24 of the first sheath 23 in the axial direction x inside the body, it is preferable to place a mark in advance at a position in the axial direction x of either the optical fiber 10 or the first sheath 23 outside the body.

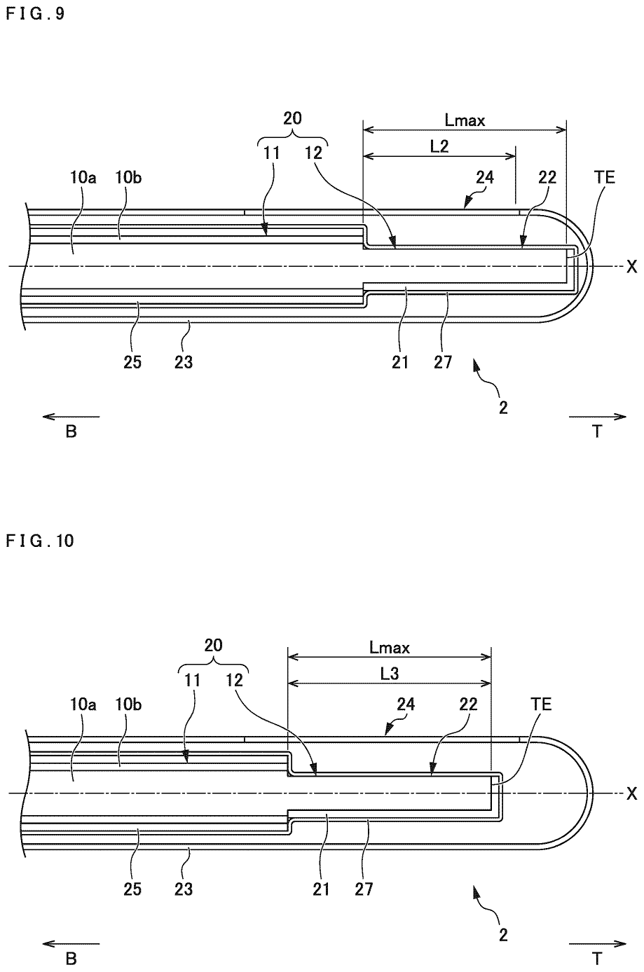

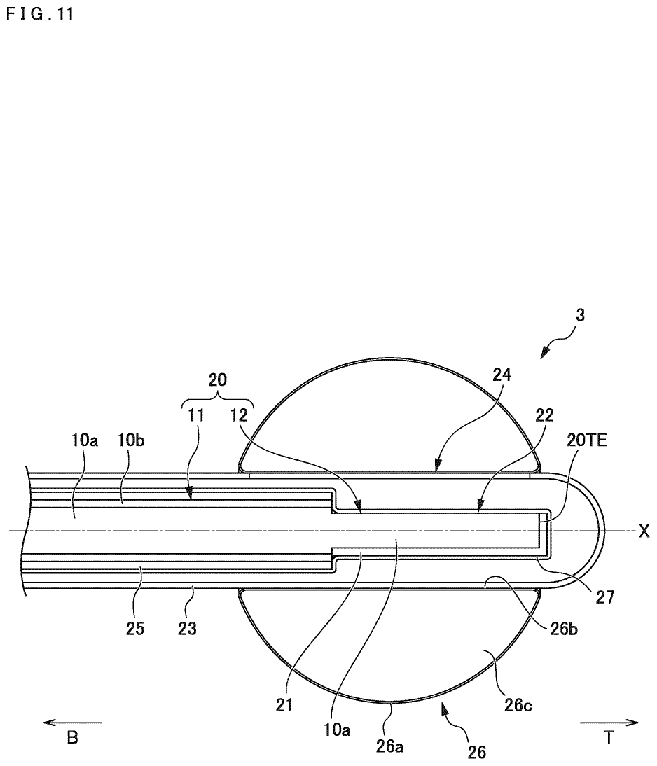

From the state illustrated in FIG. 6, when a practitioner moves the optical fiber 10 relative to the first sheath 23 toward the distal side T or the proximal side B outside the body of the subject, the light emission (irradiation) region in the axial direction x can be narrowed. FIGS. 9 and 10 are cross-sectional views of the same section as FIG. 5 and illustrate examples in which the light emission (irradiation) region in the axial direction x is altered, in the light diffusion device 2 according to the present embodiment.

FIG. 9 illustrates an example in which the optical fiber 10 is moved toward the distal side T relative to the first sheath 23 from the state illustrated in FIG. 5. In the state illustrated in FIG. 9, in the axial direction x, the light emitted from the light emission port 22 of the optical fiber 10 (within the entire region Lmax) includes light corresponding to a certain region on the distal side T, which is blocked by the first sheath 23, and only the light corresponding to the region L2 is emitted from the opening 24. Therefore, in the state illustrated in FIG. 9, the light emission (irradiation) region in the axial direction x is narrowed.

In contrast, FIG. 10 illustrates an example in which the optical fiber 10 is moved toward the proximal side B relative to the first sheath 23 from the state illustrated in FIG. 5. In the present embodiment, in the original state illustrated in FIG. 5, as can be seen from the drawing, the light emitted from the light emission port 22 of the optical fiber 10 (entire region Lmax) includes light corresponding to a very small region on the distal side T, which is blocked by the first sheath 23, and only the light corresponding to the region L1 is emitted from the opening 24.

In contrast, in the state illustrated in FIG. 10, all of the light emitted from the light emission port 22 of the optical fiber 10 in the axial direction x (entire region Lmax) is emitted from the opening 24. Therefore, in the state illustrated in FIG. 10, the light emission (irradiation) region in the axial direction x is expanded.

As described above, according to the light diffusion device 2 of the present embodiment, the first sheath 23 includes the opening 24, and the optical fiber 10 is configured to be rotatable in the circumferential direction and movable in the axial direction x relative to the first sheath 23. Thus, a practitioner can easily adjust both the width (circumferential size) and the length (axial size) of the light emission (irradiation) region.

A practitioner can manually adjust the optical fiber 10 relative to the first sheath 23 in the circumferential direction and the axial direction x. However, by providing a control mechanism including a mechanical and/or electrical structure, the practitioner can adjust the width and length of the light emission (irradiation) region more precisely and accurately.

### Third Embodiment

FIG. 11 is a cross-sectional view of a plane including an axis x of a light emission section 12 and its vicinity of the optical fiber 10 in a light diffusion device 3 according to the third embodiment; and FIG. 12 is a perspective view of the light emission section 12 and its vicinity. Since the light diffusion device 3 of the present embodiment has substantially the same configuration as the light diffusion device 1 of the first embodiment, see FIG. 1 for an overview of the light diffusion device 3.

The light diffusion device 3 according to the third embodiment has the same configuration as the light diffusion device 2 of the second embodiment, except that a second sheath 26 is added. Therefore, components having the same functions as those in the light diffusion devices 1 and 2 of the first and second embodiments, respectively, are designated with the same reference numerals as those in FIGS. 1 to 10, and detailed description thereof is omitted.

As illustrated in FIGS. 11 and 12, the light diffusion device 3 of the present embodiment includes a light-transmissive balloon-shaped second sheath 26, outside the first sheath 23. The second sheath 26 is balloon-shaped and is arranged such that the opening 24 of the first sheath 23 is positioned approximately at the center of the second sheath 26. The second sheath 26 has an overall shape resembling a flattened sphere along the axial direction x.

The second sheath 26 includes a spherical portion 26a, which is substantially spherical and externally exposed, and is formed with a through-hole 26b penetrating the center thereof in the axial direction x when viewed from one side (T side or B side) in the axial direction x. The second sheath 26 includes a sealed space 26c defined by the outer peripheral surface of the through-hole 26b and the inner surface of the spherical portion 26a, and the overall shape of the second sheath 26 is donut-shaped.

The first sheath 23 is inserted into the through-hole 26b, and the inner peripheral surface of the through-hole 26b is bonded to the outer peripheral surface of the first sheath 23 in a state where the opening 24 is positioned at a predetermined location. In the case where the interior and exterior of the first sheath 23 are not sealed or are insufficiently sealed, the sealing between the inner peripheral surface of the through-hole 26b and the outer peripheral surface of the first sheath 23 can be used to establish the seal between the interior and exterior of the first sheath 23.

The sealed space 26c is deflated while the light diffusion device 3 is being inserted into the body, and after the distal end vicinity (near the distal end T) of the light diffusion device 3 reaches a predetermined location in a body cavity, the sealed space is inflated with a liquid such as water to anchor the distal end vicinity of the light diffusion device 3 within the cavity. In this state, light may irradiate the target region while adjusting the light emission (irradiation) direction or range by the same methods as described in the first or second embodiment.

The second sheath 26 may be selected as an appropriate one from among conventionally known light-transmissive balloon-type structures having similar materials, configurations, and designs.

According to the present embodiment, even when the sealed space 26c of the second sheath 26 is filled with a liquid such as water, the interior of the first sheath 23 can be filled with air, allowing protection of the light emission port 22 and the reflective member 21 of the optical fiber 10.

The embodiments described above merely illustrate representative aspects of the present invention, and the present invention is not limited thereto. That is, those skilled in the art may implement various modifications based on conventional knowledge without departing from the spirit of the present invention. Such modifications still providing the configuration of the light diffusion device according to the present invention are, of course, included within the scope of the present invention.

For example, in the above embodiments, the shape of the light emission port is exemplified as a slit extending in the axial direction of the optical fiber; however, the present invention is not limited thereto. For example, the reflective member may be provided over the entire circumference of the light emission section, and one or more perforations may be formed on the emission-side surface of the reflective member, and such perforations may serve as the light emission port. The shape of the perforation may be arbitrary, including circular, rectangular, or any other shape.

### EXPLANATION OF REFERENCE NUMERALS

1, 2, 3: light diffusion devices
10: optical fiber
10a: core
10b: cladding
11: light transmission section
12: light emission section
12a: uneven portion
21: reflective member
22: light emission port
23: first sheath
24: opening
25: torque coil
26: second sheath
26a: spherical portion
26b: through-hole
26c: sealed space
27: protective sheath (protective member)
30: laser oscillator
BE: proximal end
TE: distal end

1. A light diffusion device, comprising:
an optical fiber, including a core located on a center side in a radial direction, and a cladding located on an outer peripheral side of the core, the light diffusion device being configured to emit, from a distal side of the optical fiber, light introduced from a proximal end portion of the optical fiber,
wherein the optical fiber is made of plastic, and includes a light transmission section configured to transmit the light from the proximal end portion to a distal end portion, and a light emission section configured to emit the light transmitted through the light transmission section from an outer peripheral surface on a distal side, the light emission section having being subjected to a treatment for suppressing heat generation,
a region other than a region serving as a light emission port on the outer peripheral surface of the light emission section, and a distal end surface, are provided with a reflective member having a light reflectance of 80% or more,
the reflective member includes light-reflective particles, and
the particles include barium sulfate.

2. The light diffusion device according to claim 1, further comprising a protective member configured to cover and seal the reflective member.

3. The light diffusion device according to claim 1, wherein at least a part of the cladding in the light emission section is removed.

4. The light diffusion device according to claim 3, wherein a rough surface is formed on an outer peripheral surface, from which the cladding in the light emission section is removed, and at least one selected from a group consisting of Rp, Rz, and Ra, which are indicators of surface roughness of the outer peripheral surface, is less than or equal to a wavelength of light emitted from the light emission section.

5. The light diffusion device according to claim 1, wherein the reflective member extending in an axial direction is provided in an arc-shaped region having a predetermined central angle θ on an outer peripheral surface of the optical fiber, and a remaining arc-shaped region not provided with the reflective member serves as the light emission port.

6. The light diffusion device according to claim 5, wherein the central angle θis 180° or more.

7. The light diffusion device according to claim 1, further comprising a light-non-transmissive first sheath configured to surround at least the light emission section of the optical fiber,
wherein an opening is provided in a region of the first sheath, such that the opening can be positioned to face the light emission port, and
the optical fiber is rotatable relative to the first sheath in a circumferential direction.

8. The light diffusion device according to claim 1, further comprising a light-non-transmissive first sheath configured to surround at least the light emission section of the optical fiber,
wherein an opening is provided in a region of the first sheath, such that the opening can be positioned to face the light emission port, and
the optical fiber is movable relative to the first sheath in an axial direction.

9. The light diffusion device according to claim 7, further comprising a light-transmissive balloon-shaped second sheath, outside the first sheath.

10. The light diffusion device according to claim 8, further comprising a light-transmissive balloon-shaped second sheath, outside the first sheath.
